(19) United States
(12) Patent Application Publication
(10) Pub. No.: US 2022/0033751 A1
(43) Pub. Date: Feb. 3, 2022

(54) Modular Bioreactor

(71) Applicant: PLURISTEM LTD., Haifa (IL)

(72) Inventors: Lior RAVIV, Kfar Monash (IL); Nadav ESHKOL, Haifa (IL); Dorina ROBERMAN, Nahariya (IL)

(73) Assignee: PLURISTEM LTD., Haifa (IL)

(21) Appl. No.: 17/276,245

(22) PCT Filed: Oct. 3, 2019
(86) PCT No.: PCT/IB2019/058429

§ 371 Date: Mar. 15, 2021

Related U.S. Application Data
(60) Provisional application No. 62/740,541, filed on Oct. 3, 2018.

(51) Int. Cl. C12M 1/00; C12M 3/00; C12M 1/12; C12M 1/34

(52) U.S. Cl. CPC C12M 23/58; C12M 23/44; C12M 41/34; C12M 41/26; C12M 25/14

(57) ABSTRACT

The present disclosure relates to use of systems for culturing, incubating, and/or expanding adherent cells.

### FIELD OF THE TECHNOLOGY

The present disclosure relates to use of systems for culturing, incubating, and/or expanding adherent cells.

### BACKGROUND

U.S. Pat. No. 6,875,605 to Teng Ma, which is incorporated by reference herein in its entirety, describes an apparatus and method for a modular cell culture bioreactor that comprises a plurality of chambers for cell culture; at least one reservoir containing a cell support medium; a plurality of conduits fluidly connecting the at least one reservoir with the plurality of chambers; and at least one pump fluidly connected through the plurality of conduits with the at least one reservoir and with the plurality of chambers to pump cell support medium therethrough; wherein each individual chamber of the plurality of chambers includes at least one three-dimensional matrix comprising polyethylene terephthalate, a plurality of channels carrying the cell support medium and having the matrix positioned in fluid communication therebetween, and at least two openings into each channel, wherein a first the opening is in fluid connection with the pump and the second opening is in fluid connection with the reservoir.

Improved incubation methods for large-scale culture and harvesting of adherent cells are urgently needed, in order to enable reliable and cost-efficient production of affordable cell-based therapies for patients in need. The present invention addresses this need.

### SUMMARY OF THE DISCLOSURE

Aspects of the disclosure relate to systems and methods that enable highly-efficient culturing, incubating, and/or expansion of adherent cells.

Additional embodiments consistent with principles of the disclosure are set forth in the detailed description which follows or may be learned by practice of methods or use of systems or articles of manufacture disclosed herein. It is understood that both the foregoing general description and the following detailed description are exemplary and explanatory only, and are not restrictive of the disclosure as claimed. Additionally, it is to be understood that other embodiments may be utilized and that electrical, logical, and structural changes may be made without departing form the spirit and scope of the present disclosure.

### BRIEF DESCRIPTION OF THE DRAWINGS

The accompanying drawings, which are incorporated in and constitute a part of this specification, illustrate several embodiments of the disclosure and, together with the description, serve to explain the principles of the disclosure. In the drawings:

FIG. 1 is a diagram of a prior art bioreactor.

FIG. 2 is a diagram of an exemplary, non-limiting modular bioreactor.

FIG. 3 is an oblique view of an exemplary cell expansion/harvest system.

FIG. 4A is an oblique view of an exemplary culture vessel. 4B-C are cutaway views of an exemplary culture vessel.

FIG. 5 is an exploded view of certain components of a lower portion of an exemplary culture vessel.

FIG. 6 is an exploded view of certain components of an upper portion of an exemplary culture vessel.

### DESCRIPTION OF THE EMBODIMENTS

Reference will now be made in detail to the present embodiments of the disclosure, examples of which are illustrated in the accompanying drawings.

In this application, the use of the singular includes the plural unless specifically stated otherwise. Also in this application, the use of “or” means “and/or” unless stated otherwise. Furthermore, the use of the term “including,” as well as other forms, such as “includes” and “included,” are not limiting. Any range described herein will be understood to include the endpoints and all values between the end points.

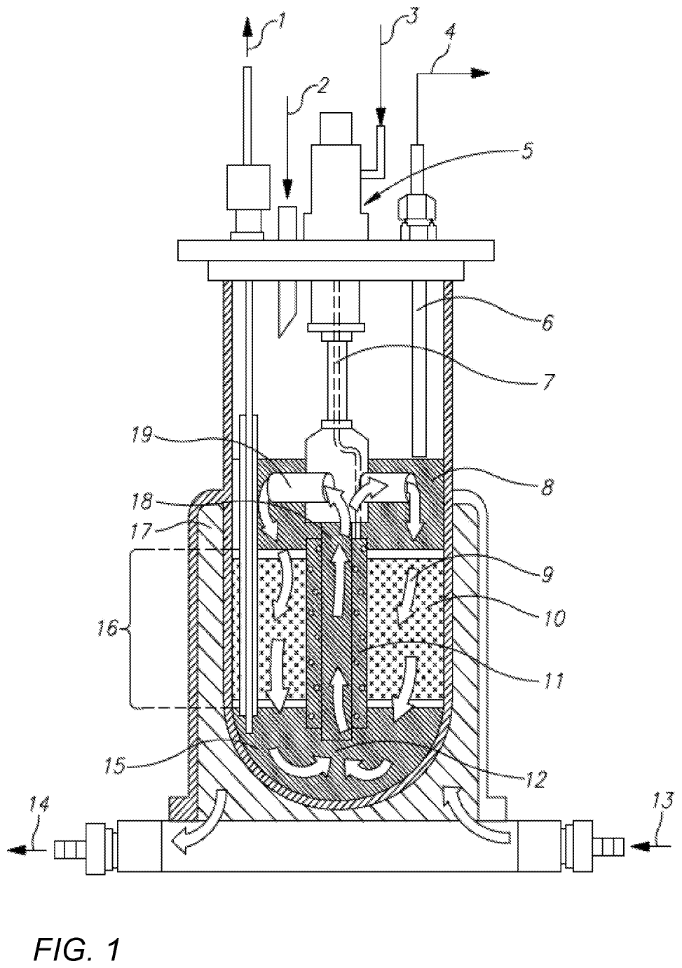

A prior art bioreactor, the Celligen 310 Bioreactor, is depicted in FIG. 1. A Fibrous-Bed Basket (16) is loaded with polyester disks (10). The vessel is filled with deionized water or isotonic buffer via an external port (1) that is used for cell harvesting and then autoclaved. Following sterilization, the liquid is replaced with growth medium, which saturates the disk bed as depicted in (9). Temperature, pH, dissolved oxygen concentration, etc., are set prior to inoculation. A slow initial stiffing rate is used to promote cell attachment, then the stirring rate is increased. Perfusion is initiated by adding fresh medium via an external port (2). If desired, metabolic products may be harvested from the cell-free medium above the basket (8). Rotation of the impeller creates negative pressure in the draft-tube (18), which pulls cell-free effluent from a lower region (15) through the draft tube, then through an impeller port (19), thus causing medium to circulate (12) uniformly in a continuous loop. Adjustment of a tube (6) controls the liquid level; an external opening (4) of this tube is used for harvesting. A ring sparger (not visible), is located inside the impeller aeration chamber (11), for oxygenating the medium flowing through the impeller, via gases added from an external port (3), which may be kept inside a housing (5), and a sparger line (7). Sparged gas confined to the remote chamber is absorbed by the nutrient medium, which washes over the immobilized cells. Water jacket (17) contains ports for moving the jacket water in (13) and out (14).

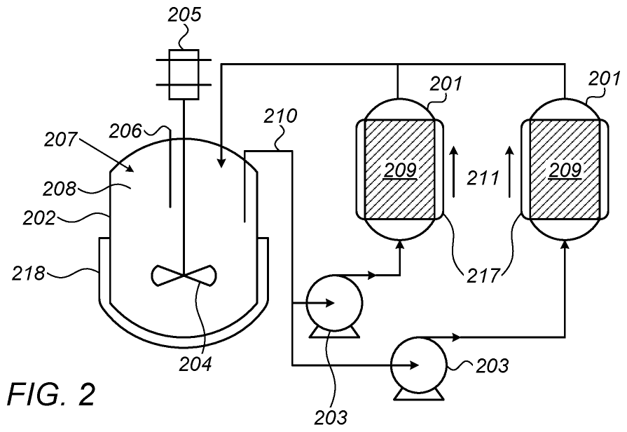

Provided herein, in certain embodiments, is a modular cell culture apparatus whose schematic is shown in FIG. 2, comprising: (a) a central medium container (a.k.a. reservoir) 202, comprising a cell culture medium (not depicted), wherein central medium container 202 does not contain cells; and (b) a plurality of 3-D culture vessels 201, wherein each of the culture vessels 201 comprises microcarriers composed of a 3-D substrate 209, e.g. a fibrous matrix, which may be, in some embodiments, a synthetic matrix; wherein the central medium container 202 is operably connected (e.g. via tubing 210) to the culture vessels 201, such that medium from the medium container 202 flows through the vessels 201 in parallel, or, in more specific embodiments, through said microcarriers, in other embodiments, through said 3-D substrate of said microcarriers. In certain embodiments, the flow is against gravity. More specifically, the vessels may be oriented vertically, with the flow in an upward direction. In other embodiments, a plurality of cell culture carriers (not depicted) composed of 3-D substrate 209 are disposed within each of culture vessels 201. Alternatively or in addition, the apparatus is aseptically sealed. In other embodiments, the described apparatus is a closed system.

Preferably, central medium container 202 does not comprise a cell culture substrate. Optionally, the apparatus further comprises one or more circulation pumps 203 or other means of actively transporting the medium through the vessels 201. In certain embodiments, there is one pump 203 operably connected to each vessel 201. In various embodiments, the vessels 201 are not directly physically connected with one another; or at least one of the vessels 201 is directly physically connected with one or more other vessels 201. Connection via tubing 210 and/or the central medium container 202 is, naturally, not considered direct physical connection in this regard.

In yet other embodiments, each of the plurality of vessels 201 is temperature-insulated. Alternatively or in addition, central medium container 202 is temperature-insulated. Non-limiting examples of temperature insulation are medium container water jacket 218 and vessel water jacket 217, which may be independently various types of water jackets known in the art. In still other embodiments, both central medium container 2 and culture vessels 1 are temperature controlled, or, in other embodiments, are operably connected with a thermometer, thermostat, and/or other means for controlling the temperatures of the fluid contents thereof.

The term “3-D culture vessel(s)”, as used herein, refers to a vessel (e.g. as depicted in 201) configured to hold a liquid medium and a 3D substrate 209. Preferably, vessel 201 is further configured to be aseptically sealed.

In certain embodiments, the cells in the described vessels are adhered to 3D carriers, which refers to carriers that facilitate 3D culture (as defined herein). The carriers may be, in more specific embodiments, selected from macrocarriers, microcarriers, or either. Non-limiting examples of microcarriers that are available commercially include alginate-based (GEM, Global Cell Solutions), dextran-based (Cytodex, GE Healthcare), collagen-based (Cultispher, Percell), and polystyrene-based (SoloHill Engineering) microcarriers. In certain embodiments, the microcarriers are packed inside the vessels. In other embodiments, the 3D carriers are fibrous 3D carriers that comprise an adherent material, which may be, in more specific embodiments, microcarriers that are 100-10,000 microns in diameter (measured along the largest dimension, when non-spherical), or, in other embodiments, 100-8,000, 100-6,000, 200-10,000, 200-8,000, 200-6,000, 300-10,000, 300-8,000, 300-6,000, 500-10,000, 500-8,000, 500-6,000, 800-10,000, 800-8,000, or 800-6,000 microns.

Medium container 202 may contain a mixing device, which may be e.g. an impeller 204, which is driven by agitation motor 205 and mixes the medium within medium container 202. Those skilled in the art will appreciate, in light of the present disclosure, that suitable mixing devices include, but are not limited to, marine-blade impellers, pitched-bladed impellers (e.g. high-solidity pitch-blade impellers), hydrofoil impellers (e.g. high-solidity hydrofoil impellers), Rushton impellers, pitched-blade impellers, CelliGen® cell-lift impeller, A320 Impeller (SPX Flow), HE3 Impeller (Chemineer), and the like.

Medium container 202 is also optionally connected with one or more control loops 206, for monitoring and controlling pH, dissolved oxygen concentration, and temperature; feed line 207, for introducing fresh medium to the medium container 202, and waste line 208, for removing spent medium from medium container 202. Preferably, perfusion involves the functions of both feed line 207 and waste line 208. Control loops 206 may include a pH adjustment solution line (not depicted), for introducing basic or acidic solution, as necessary to modulate pH.

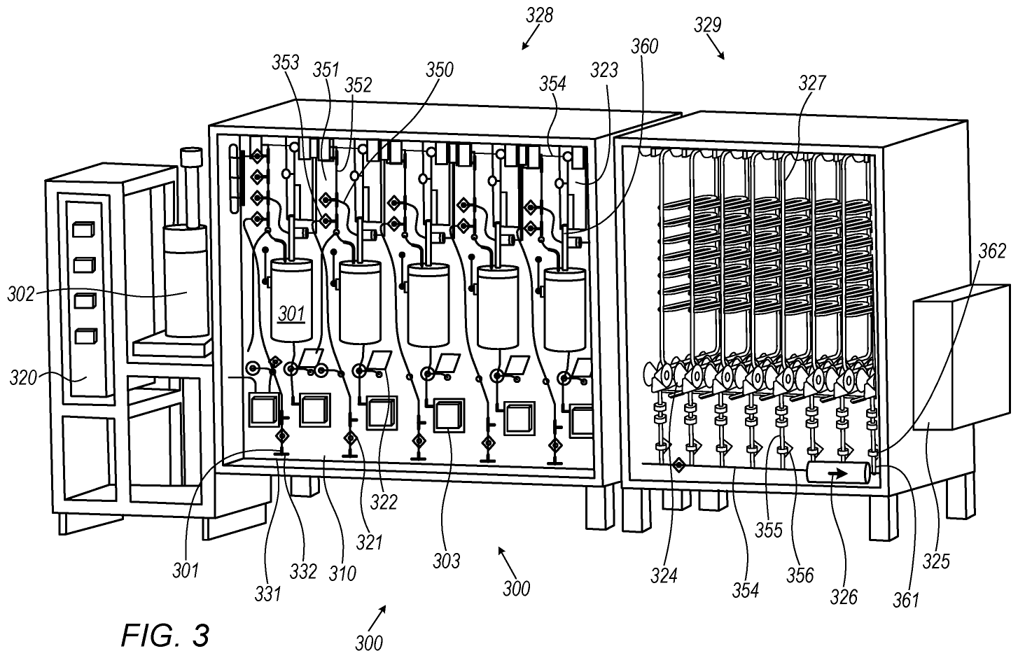

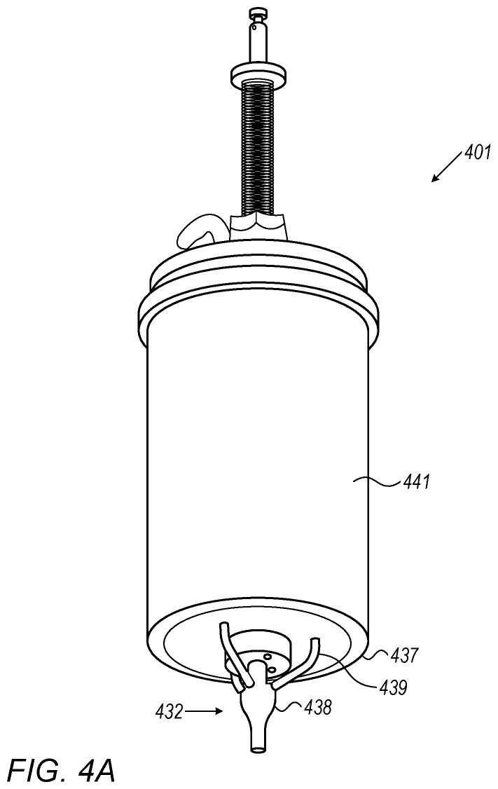

Further aspects are depicted in FIG. 3. Cell expansion/harvest system 300 contains tower 320, central medium container (a.k.a. reservoir) 302, expansion/harvest module 328, harvest bag module 329, and electrical cabinet 325. Tower 320 houses pumps (not depicted) for feed, waste and basic solution (for adjusting pH of the medium). Central medium container 302 houses growth medium (not depicted). Expansion/harvest module 328 houses culture vessels 301, harvest motor 323, and associated tubing and valves (described below). Harvest bag module 329 houses lattice 327, solution/harvest bag scales 324, solution and harvest bags (not depicted), filter 326 for post-harvest filtration, and associated tubing and valves (described below). Growth medium from central medium container 302 flows through central medium tube 310, through inflow branch points 331 and inflow branch tubes 332, into culture vessels 301, each of which contains an inner compartment (e.g. a basket [see FIG. 4B-C]). Inflow branch tubes 332 may be operably connected with medium pumps 303, inflow pinch valves 321, and/or flow meters 322. In more specific embodiments, growth medium flows through said 3-D substrate.

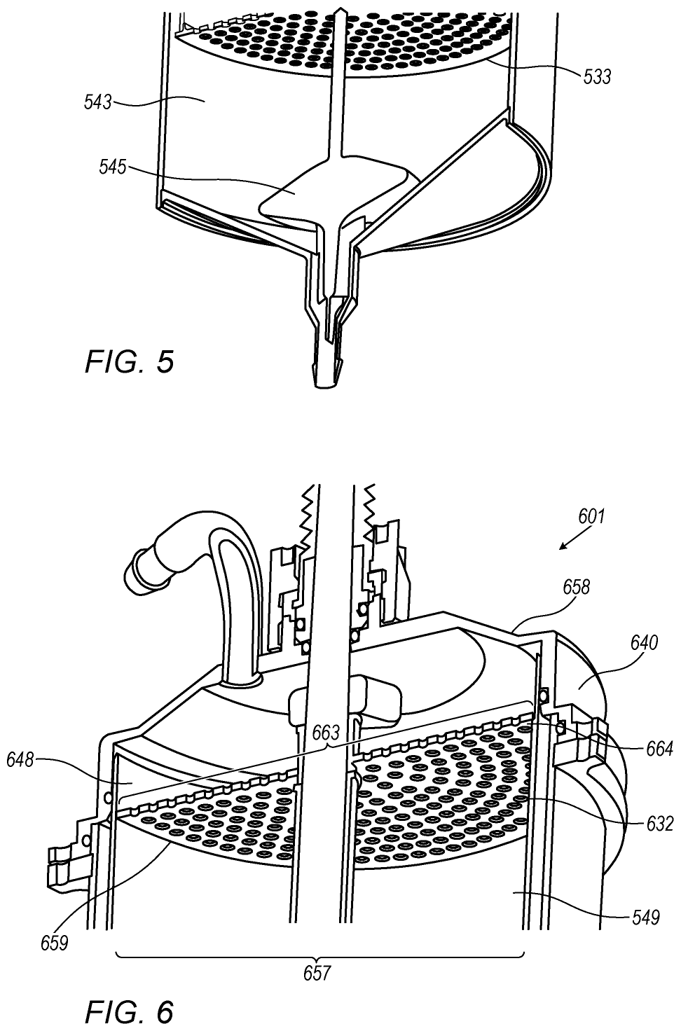

Waste medium from culture vessels 301 flows through outflow branch tubes 350 and outflow pinch valves 351, into central waste tube (not shown). Harvest motor 323 enables a harvesting process that comprises oscillation, without the need to move culture vessels 301 to a separate housing. Harvest solution(s) (not depicted) flow from solution bags through solution branch tubes 355 and solution pinch valves 356 into central solution/harvest tube 354, which bridges harvest bag module 329 and expansion/harvest module 328. Central solution/harvest tube 354 also connects to solution/harvest branch tubes 352 and solution/harvest pinch valves 353, allowing harvest solutions to enter and exit culture vessels 301. Harvest motor 323 connects to basket (see FIG. 4B-C) via connecting shaft 360, which transects top side 658 of culture vessel 601 (see FIG. 6), enabling oscillating of basket, optionally in the presence of harvest solutions. Following solution exposure and oscillation, cell suspension (not depicted) flows through solution/harvest pinch valves 353 and solution/harvest branch tubes 352, into central solution/harvest tube 354, which leads to filter 326; which in turn leads to harvest branch tubes 361, harvest pinch valves 362, and harvest bag(s) (not depicted), which optionally are pre-loaded with enzyme neutralization solution, which (branch tubes 361, pinch valves 362, and harvest bags) are disposed distal to filter 326.

In certain embodiments, vessels 301 do not comprise control loops or mixing devices (e.g. impellers and the like). Applicant has realized that the absence of mixing devices and control loops attached to vessels enables harvest motor 323 to be readily co-localized with vessels 301 inside expansion/harvest module 328, significantly decreasing the footprint of the described cell expansion/harvest process.

Harvest solutions, as used herein, refers to any buffered rinse solution (e.g. isotonic buffer or the like), protease or enzyme solution (non-limiting examples of which are found in in PCT International Application Publ. No. WO 2012/140519, which is incorporated herein by reference), or neutralization solution (e.g. complete medium or the like) useful in removal of adherent cells from a substrate. Those skilled in the art will ready ascertain what solutions fall under this classification.

In certain embodiments, as depicted in FIG. 4A, growth medium flows into culture vessel 401 via a lower plate 437 that delineates the bottom of culture vessel 401, generating an upward pressure and resulting in upward flow of growth medium. Inflow branch tube 432 leads into tube junction 438, which splits flow into sub-flow tubes 439, which lead into interior 441 of culture vessel 401 via perforations (not depicted) in lower plate 437. FIG. 4B shows a cutaway view of culture vessel 401, the interior of which is partially occupied by basket 431, which holds 3D carriers (not depicted). Sub-flow tubes 439 are typically flush with lower plate 437, but may also optionally slightly protrude (typically less than 1 centimeter) through lower plate 437 into interior 441 of culture vessel 401. Culture vessel 401 may further include basket positioning pin 436, which may mate with hollow central axis 435 of basket 431. Those skilled in the art will appreciate that, while 3 sub-flow tubes 439 are depicted, having apertures in a triangular configuration, use of different numbers and configurations of sub-flow tubes (e.g. 2, 4, 5, 6, 2-4, 2-5, 2-6, 3-4, 3-5, 3-6, 2-8, or 3-8) is consistent with the present disclosure. Typically, 3-6 sub-flow tubes are utilized. Applicant has realized that mixing of growth medium prior to its entry into basket carries advantages.

FIG. 4C shows an aspect wherein growth medium flows into culture vessel 401 via single aperture 442. Flow disruptor 445 is disposed distal to aperture 442, which also achieves relative homogeneity of growth medium within lower space 443, prior to entry into basket 431. Lower and upper boundaries of basket 431 are defined by lower screen 433 and upper screen 432, respectively. Interior basket space 444 is optionally subdivided by intermediate screens 434.

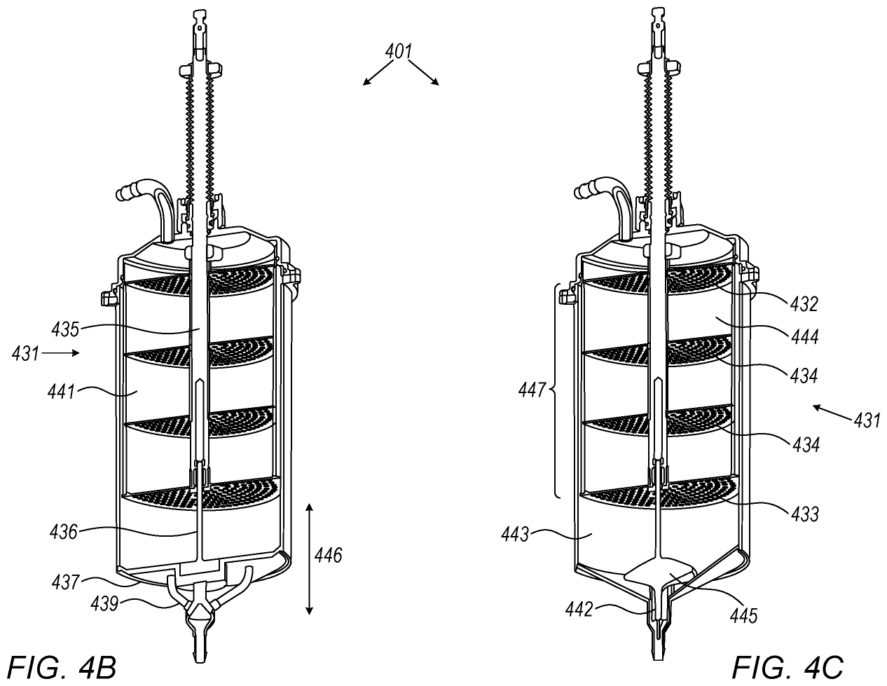

FIG. 5 shows an exploded view of flow disruptor 545, lower space 543 of culture vessels (partially depicted), and lower screen 533 of basket (partially depicted).

FIG. 6 depicts an aspect wherein basket (partially depicted) and culture vessel 601 are configured to jointly form a seal between a perimeter 659 of basket and an inner surface 649 of culture vessel 601. For example, upper screen 632 of basket forms a seal with side wall 648 at point 640 wherein inner diameter 657 of culture vessel 601 narrows proximal to top side 658 of culture vessel 601. In more specific embodiments, lower inner diameter 657 of culture vessel 601 is greater than diameter 663 of basket, enabling oscillation of basket within culture vessels 601. In certain embodiments, basket is locked into upper position to form a watertight seal. Applicant has realized that a watertight seal between a perimeter 659 of basket and an inner surface 649 of culture vessel 601, combined with upward flow of growth medium, causes culture medium present in culture vessel 601 to preferentially pass through basket, thus improving perfusion of 3D carriers disposed within basket, and cells associated with 3D carriers. Upper screen 632 and lower screen 533 of basket contain apertures 664, allowing passage of medium and other fluids therethrough.

For harvest, basket 431 is oscillated within (and relative to) culture vessel 401, along longitudinal axis 446 of culture vessel 401.

Adherent cells can be propagated, in some embodiments, by using a combination of 2D and 3D substrates, e.g. prior to and in conjunction with the disclosed modular bioreactor, respectively; using suitable growth medium/media known in the art. The term medium, except where indicated otherwise, refers to a liquid composition designed for ex-vivo replication (“tissue culture”) of adherent cells. Further, non-limiting examples of suitable media are mentioned herein.

Reference herein to “growth” of a population of cells is intended to be synonymous with expansion of a cell population. In certain embodiments, ASC (which may be, in certain embodiments, placental ASC), are expanded without substantial differentiation. In various embodiments, the described expansion is on a 2D substrate, followed by a 3D substrate.

In other embodiments, there is a provided a method of culturing adherent cells, comprising expanded cells in the described apparatus. In certain embodiments, culturing in the described apparatus is preceded by 2D culturing. Any described embodiments of the apparatus may apply to the culturing methods.

The terms “two-dimensional culture” and “2D culture” refer to a culture in which the cells are exposed to conditions that are compatible with cell growth and allow the cells to grow in a monolayer. An apparatus suitable for such are is referred to as a “2D culture apparatus”. Such apparatuses will typically have flat growth surfaces (also referred to as a “two-dimensional substrate(s)” or “2D substrate(s)”), in some embodiments comprising an adherent material, which may be flat or curved. Non-limiting examples of apparatuses for 2D culture are cell culture dishes and plates. Included in this definition are multi-layer trays, such as Cell Factory™, manufactured by Nunc™, provided that each layer supports monolayer culture. It will be appreciated that even in 2D apparatuses, cells can grow over one another when allowed to become over-confluent. This does not affect the classification of the apparatus as “two-dimensional”. In certain embodiments, 2D culture is performed prior to culturing cells in the described modular apparatus.

The terms “three-dimensional culture” and “3D culture” refer to a culture in which the cells are exposed to conditions that are compatible with cell growth and allow the cells to grow in a 3D orientation relative to one another. Such conditions will typically utilize a 3D growth surface (also referred to as a “three-dimensional substrate” or “3D substrate”), in some embodiments comprising an adherent material, which is present in the 3D culture vessels. Certain, non-limiting embodiments of 3D substrates suitable for expansion of ASC are described in PCT Application Publ. No. WO/2007/108003, which is fully incorporated herein by reference in its entirety. Preferably, 3D culture is performed in conjunction with the described modular apparatus.

In certain embodiments, the systems described herein are closed systems. Alternatively or in addition, the described processes are automated processes. Those skilled in the art will appreciate in light of the present disclosure that closed systems are sealed from the outside environment, in a manner enabling maintenance of sterility. In further embodiments, closed systems are sealed in a manner preventing unintentional contamination by substances outside the system. In yet other embodiments, closed systems are sealed in an airtight manner. The skilled person will further appreciate that closed systems enable manipulation of the contents thereof without requiring the manipulation to take place inside a sterile hood or sterile room.

In other, optional embodiments, any of the described methods further comprises determining the concentration of cells in the vessels. Thus, the described vessel(s) is/are optionally further operably connected to a sensor for determining the cell concentration. In more specific embodiments, the cells may be adherent stromal cells (ASC). In yet more specific embodiments, the ASC are placenta-derived. Alternatively, the ASC are derived from adipose tissue; or in other embodiments, from bone marrow; or, in other embodiments, from another suitable tissue source; e.g. peripheral blood; umbilical cord blood; synovial fluid; synovial membranes; spleen; thymus; mucosa (for example nasal mucosa); limbal stroma; a ligament (for example the periodontal ligament); scalp; hair follicles, testicles; embryonic yolk sac; and amniotic fluid.

In still other embodiments, any of the described methods further comprises measuring viability of cells in the vessels. In other embodiments, the described apparatus further comprises a probe, or other means of measuring viability of cells in the vessels.

In yet other embodiments, any of the described methods further comprises monitoring and/or controlling pH of the medium in central medium container. In other embodiments, the described apparatus comprises a measuring device and/or input channel for monitoring and/or controlling pH of the medium. Those skilled in the art will appreciate, in light of the present disclosure, that the pH of a liquid formulation can be adjusted in a variety of ways known in the art, non-limiting examples of which are addition of carbon dioxide (CO₂), base solution, acid solution, and/or pH buffer to the formulation. Non-limiting examples of means for adjusting pH include input channels and pumps for addition of CO₂, base solution, acid solution, and/or pH buffer to the formulation. In certain embodiments, the described system comprises adjustable controls for the pH of the formulation.

In other embodiments, any of the described methods further comprises monitoring and/or controlling the dissolved oxygen concentration (pO₂) inside the medium container, or in other embodiments, the vessels, or in other embodiments, both the medium container and the vessels. In other embodiments, the apparatus may further comprise a meter or other means of monitoring and/or controlling the dissolved oxygen concentration inside medium container. pO₂ can be adjusted (as a non-limiting example) by addition of O₂ to a formulation, in some embodiments using a pump. In certain embodiments, the described system comprises adjustable controls for the pO₂ of the medium inside the medium container. In still other embodiments, measurement of pO₂ serves to estimate the number of viable cells in the vessels.

In other embodiments, any of the described methods further comprises monitoring and/or controlling the temperature of medium inside the medium container, or in other embodiments, the vessels, or in other embodiments, both the medium container and the vessels. Thus, the apparatus may further comprise a thermometer, thermostat, or other means of monitoring and/or controlling the temperature of medium inside the medium container and/or the vessels, non-limiting examples of which are thermometers, insulation, and external containers for a fluid, e.g. a liquid or a gas, whose temperature can be manipulated. Methods for determining and adjusting temperature of a medium are well known in the art. In certain embodiments, the described system comprises adjustable controls for the temperature of the medium.

In yet other embodiments, any of the described methods further comprises collecting and/or storing data on conditions inside the medium container, which may be, e.g. glucose concentration, temperature, pH, dissolved oxygen concentration, etc. In other embodiments, the apparatus optionally further comprises a meter(s), connection to an external computer, and/or other means of collecting and/or storing data on conditions inside the medium container. In certain embodiments, the data is used to generate a report.

In still other embodiments, any of the described methods further comprises collecting and/or storing data on transfer of fluid into and/or out of the medium container, or in other embodiments, into or out of the vessels, or in still other embodiments, both the medium container and the vessels. In other embodiments, the apparatus optionally further comprises a meter(s), connection to an external computer, and/or other means of collecting and/or storing data on transfer of fluid into and/or out of the medium container and/or the vessels. In certain embodiments, the data is used to generate a report.

In yet other embodiments, any of the described methods further comprises controlling the flow rate of medium transferred into and/or out of the central medium container, or in other embodiments, into and/or out of the culture vessels, or in still other embodiments, both the medium container and the vessels. In other embodiments, the apparatus optionally further comprises a meter(s), connection to an external computer, and/or other means of controlling a flow rate of medium transferred into, and/or out of, each of the culture vessels.

In yet other embodiments, any of the described methods further comprises facilitating uniform mixing of liquid contents of the described medium container when a stirrer/agitation device is activated (e.g. rotated). Thus, the medium container optionally further comprises one baffle or, in other embodiments more than one baffles, that jut(s) inward from an inward surface of the container.

In still other embodiments, the described medium container is, optionally, further operably connected to an automatic calibrator and/or other means of calibrating other components and/or sensors described herein and/or monitoring the failure of one, some, or all of these components, of which represents a separate embodiment.

Each of the described optional method steps and optional components represents a separate embodiment, and they may be freely combined, in various embodiments.

In certain embodiments, the described methods and systems are aseptic.

Also provided herein is an enclosed system, comprising a cell culture apparatus, comprising: (a) a central medium container, comprising a cell culture medium, wherein the central medium container does not contain cells; and (b) a plurality of 3-D culture vessels, wherein each culture vessel comprises a plurality of cell carriers, said carriers comprising a 3-D matrix, e.g. a fibrous matrix, which may be, in some embodiments, a synthetic matrix; wherein the central medium container is operably connected to the culture vessels, such that medium from the medium container flows through the 3-D matrices of the vessels in parallel, after reaching the vessels via suitable conduits. Optionally, the apparatus further comprises a pump or other means of actively transporting the medium through the vessels. In other embodiments, the described apparatus is a closed system. Alternatively or additionally, the apparatus is configured to circulate medium through the vessels against the force of gravity, i.e. from the bottom towards the top of the vessels. For example, in the case of a vertical-oriented, cylindrical vessel, the medium is, in certain embodiments, introduced into the bottom of the vessel and exits through the top thereof (indicated in FIG. 2 as 211). “Vertical”, as used herein, encompasses configurations where the referred to component, e.g. the long axis of a cylindrical vessel, is oriented approximately vertically, e.g. within 30 degrees, or, in other embodiments, 25, 20, 18, 15, 12, 10, 8, 6, or 5 degrees (on a 360 degree scale) of absolute verticality. In more specific embodiments, the flow of fluid through the described cylindrical vessel is parallel to its long axis, and may be against the flow of gravity (upward).

Except where indicated otherwise, the term enclosed system indicates that the internal space of the system is encased so as to be physically separated from outside contaminants. Those skilled in the art will appreciate in light of the present disclosure that enclosed systems may, in some embodiments, comprise a closed volume and/or be sealed from the outside environment, in a manner enabling maintenance of sterility. In further embodiments, enclosed systems are sealed in a manner preventing unintentional contamination by substances outside the system. In yet other embodiments, enclosed systems are sealed in an airtight manner. The skilled person will further appreciate that enclosed systems enable manipulation of the contents thereof (e.g. perfusion of the system with solution from an external tank feed, circulation of the medium within the system, and removal of waste buffer into a waste container), without requiring the work to take place inside a sterile hood or other sterile environment.

In certain embodiments, the described apparatus is configured for seeding the cells on the fibrous matrix contained within the vessels. In other embodiments, the described method comprises seeding the cells on the fibrous matrix contained within the vessels. In more specific embodiments, the seeding method comprises flowing a cell suspension through the vessels against the direction of gravity.

In other embodiments, there is provided a method of seeding cells in a modular bioreactor, comprising flowing a cell suspension through the vessels against the direction of gravity. Any described embodiments of the modular bioreactor may apply to this method.

In certain embodiments, any of the described systems is configured for, and/or is capable of, cell culture, i.e. ex-vivo expansion of cells. In other embodiments, the cells are ASC, non-limiting examples of which are placental ASC, adipose ASC, and bone-marrow (BM)-derived ASC. In other embodiments, the cells are mesenchymal stromal cells (MSC).

In still other embodiments, the described culture vessel(s) is, optionally, further operably connected to a sensor for determining an average size of cells in the vessels, which may be, in non-limiting embodiments, living cells, or in other embodiments, inactivated cells.

In other embodiments, any of the described systems, optionally, further comprises a pump or other means of controlling a flow rate of a fluid material perfused into, and/or, in other embodiments, removed from, each of the vessels.

Each of the described embodiments of the features of the central medium container, vessels, and/or means of fluid transport may be freely combined with each other. Moreover, each of these embodiments may be freely combined with each of the basic bioreactor embodiments described herein, including those depicted in FIG. 1.

In some embodiments, the carriers in the vessels are loosely packed, for example forming a loose packed bed, which is submerged in a nutrient medium. Alternatively or in addition, the 3D carriers are fibrous 3D carriers, which are typically deformable and comprise a cell-adherent material (“adherent material”). In other embodiments, the surface of the carriers comprises an adherent material, or the surface of the carriers is adherent. In still other embodiments, the material exhibits a chemical structure such as charged surface exposed groups, which allows cell adhesion. Non-limiting examples of adherent materials which may be used in accordance with this aspect include a polyester, a polypropylene, a polyalkylene, a polyfluorochloroethylene, a polyvinyl chloride, a polystyrene, a polysulfone, a cellulose acetate, a glass fiber, a ceramic particle, a poly-L-lactic acid, and an inert metal fiber. In more particular embodiments, the material may be selected from a polyester and a polypropylene. In various embodiments, an “adherent material” refers to a material that is synthetic, or in other embodiments naturally occurring, or in other embodiments a combination thereof. In certain embodiments, the material is non-cytotoxic (or, in other embodiments, is biologically compatible). Non-limiting examples of synthetic adherent materials include polyesters, polypropylenes, polyalkylenes, polyfluorochloroethylenes, polyvinyl chlorides, polystyrenes, polysulfones, cellulose acetates, and poly-L-lactic acids, glass fibers, ceramic particles, and an inert metal fiber, or, in more specific embodiments, polyesters, polypropylenes, polyalkylenes, polyfluorochloroethylenes, polyvinyl chlorides, polystyrenes, polysulfones, cellulose acetates, and poly-L-lactic acids. Other embodiments include Matrigel™, an extra-cellular matrix component (e.g., Fibronectin, Chondronectin, Laminin), and a collagen. In certain embodiments, flow through the described vessels is directed to pass through a bed of fibrous carriers. Applicant has realized that use of deformable carriers facilitates harvest using the described modular systems.

In certain embodiments, the cells in the vessels are subjected, following expansion, to a harvesting process that comprises oscillation. In certain embodiments, the agitation is vibration, for example as described in PCT International Application Publ. No. WO 2012/140519, which is incorporated herein by reference. Typically, basket 431 is disposed within culture vessel 401 and oscillated within (and relative to) culture vessel 401. In certain embodiments, basket is subdivided by intermediate screen(s) 434 into subsections. When present, intermediate screen(s) 434 contain apertures 664, allowing passage of medium and other fluids therethrough.

Screen(s), as used herein, refers to a flat structure containing apertures of sufficient width to permit passage of fluid at ambient pressure. Preferably, width of apertures is not sufficient to enable passage of cell carriers therethrough.

In other embodiments, there is provided a method for harvesting cells within a parallel, modular cell culture apparatus, comprising oscillating an inner container comprising upper, intermediate, and lower screens, relative to an outer vessel, within which said inner container is disposed. In still other embodiments, there is a provided a harvest apparatus within a parallel, modular cell culture system, comprising an inner container comprising upper, intermediate, and lower screens; wherein said apparatus is configured to oscillate said inner container relative to an outer vessel, within which said inner container is disposed. Any described embodiments of the modular bioreactor may apply to this method.

In certain embodiments, during harvesting, the basket is agitated at 0.4-6 Hertz (24-360 oscillations per minute), in other embodiments 0.7-6 Hertz, in other embodiments 1-6 Hertz, in other embodiments 0.7-3 Hertz, in other embodiments 1-5 Hertz, in other embodiments 2-5 Hertz, in other embodiments 1-4 Hertz, or in other embodiments 1-3 Hertz, during, or in other embodiments during and after, treatment with a protease, optionally also comprising a calcium chelator. In certain embodiments, a basket containing fibrous carriers is agitated at 0.4-6 Hertz, 0.7-6 Hertz, 1-6 Hertz, 0.7-3 Hertz, 1-5 Hertz, 2-5 Hertz, 1-4 Hertz, or in other embodiments 1-3 Hertz, while submerged in a solution or medium comprising a protease, optionally also comprising a calcium chelator. Non-limiting examples of a protease plus a calcium chelator are trypsin, or another enzyme with similar activity, optionally in combination with another enzyme, non-limiting examples of which are Collagenase Types I, II, III, and IV, with EDTA. Enzymes with similar activity to trypsin are well known in the art; non-limiting examples are TrypLE™, a fungal trypsin-like protease, and Collagenase Types I, II, III, and IV, which are available commercially from Life Technologies. Enzymes with similar activity to collagenase are well known in the art; non-limiting examples are Dispase I and Dispase II, which are available commercially from Sigma-Aldrich. In still other embodiments, the cells are harvested by a process comprising an optional wash step, followed by optional incubation with collagenase, followed by incubation with trypsin under oscillation. Alternatively or in addition, the enzyme solution is replaced by a wash solution before removing the cells via oscillation. In various embodiments, at least one, at least two, or all three of the aforementioned steps comprise agitation. In more specific embodiments, cells are removed from culture vessels simultaneously.

Alternatively or in addition, the ASC are expanded using an adherent material in a basket, which is in turn disposed within a bioreactor chamber (corresponding to the described culture vessels); and an apparatus is used to impart a reciprocating motion to the basket relative to the bioreactor chamber, wherein the apparatus is configured to move the basket in a manner causing cells attached to the adherent material to detach from the adherent material. In more specific embodiments, the vibrator comprises one or more controls for adjusting amplitude and frequency of the reciprocating motion. Alternatively or in addition, the adherent material is a 3D substrate, which comprises, in some embodiments, carriers comprising a synthetic adherent material.

In still other embodiments, adherent cells are passaged within the bioreactor by harvesting the cells from the carriers (e.g. as described herein), thus forming a cell suspension within the described system. In further embodiments, the cell suspension is seeded on additional carriers in the same system or a new system. In still other embodiments, the passaging is performed in an aseptic manner.

Those skilled in the art will appreciate that a variety of isotonic buffers and media may be used in the described methods and systems. Hank's Balanced Salt Solution (HMS; Life Technologies) is only one of many buffers that may be used. Other, non-limiting examples of useful base media include Minimum Essential Medium Eagle, ADC-1, LPM (Bovine Serum Albumin-free), F10 (HAM), F12 (HAM), DCCM1, DCCM2, RPMI 1640, BGJ Medium (with and without Fitton-Jackson Modification), Basal Medium Eagle (BME-with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E-with Earle's sale base), Medium M199 (M199H-with Hank's salt base), Minimum Essential Medium Eagle (MEM-E-with Earle's salt base), Minimum Essential Medium Eagle (MEM-H-with Hank's salt base) and Minimum Essential Medium Eagle (MEM-NAA with non-essential amino acids), among numerous others, including medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, MDBC 153. In certain embodiments, DMEM is used. These and other useful media are available from GIBCO, Grand Island, N.Y., USA and Biological Industries, Bet HaEmek, Israel, among others.

In some embodiments, the medium may be supplemented with additional substances. Non-limiting examples of such substances are serum, which is, in some embodiments, fetal serum of cows or other species, which is, in some embodiments, 5-15% of the medium volume. In certain embodiments, the medium contains 1-5%, 2-5%, 3-5%, 1-10%, 2-10%, 3-10%, 4-15%, 5-14%, 6-14%, 6-13%, 7-13%, 8-12%, 8-13%, 9-12%, 9-11%, or 9.5%-10.5% serum, which may be fetal bovine serum, or in other embodiments another animal serum. In still other embodiments, the medium is serum-free.

Alternatively or in addition, the medium may be supplemented by growth factors, vitamins (e.g. ascorbic acid), cytokines, salts (e.g. B-glycerophosphate), steroids (e.g. dexamethasone) and hormones e.g., growth hormone, erythropoietin, thrombopoietin, interleukin 3, interleukin 7, macrophage colony stimulating factor, c-kit ligand/stem cell factor, osteoprotegerin ligand, insulin, insulin-like growth factor, epidermal growth factor, fibroblast growth factor, nerve growth factor, ciliary neurotrophic factor, platelet-derived growth factor, and bone morphogenetic protein.

It will be appreciated that additional components may be added to the culture medium. Such components may be antibiotics, antimycotics, albumin, amino acids, and other components known to the art for the culture of cells.

It will also be appreciated that in certain embodiments, when the described ASC are intended for administration to a human subject, the cells and the culture medium (e.g., with the above-described medium additives) are substantially xeno-free, i.e., devoid of any animal contaminants e.g., mycoplasma. For example, the culture medium can be supplemented with a serum-replacement, human serum and/or synthetic or recombinantly produced factors.

In certain embodiments, the described systems and methods enable conservation of medium. The medium consumption is, in some embodiments, significantly less than would be used in a “scale-out” expansion of a traditional bioreactor, where the tank holding the medium and cell carriers are the same vessel. Alternatively or in addition, the systems and methods enable use of a smaller area than prior art systems.

In certain embodiments, the total volume of medium used in the described methods and systems is at least 25 liters, at least 30 liters, at least 35 liters, at least 40 liters, at least 50 liters, at least 70 liters, at least 100 liters, at least 150 liters, at least 200 liters, at least 300 liters, at least 500 liters, between 25-300 liters, between 25-500 liters, between 30-300 liters, between 30-500 liters, between 40-300 liters, between 40-500 liters, between 50-300 liters, between 50-500 liters, between 100-300 liters, or between 100-500 liters. In still other embodiments, not less than about 23 liters of medium (e.g. 8-23, 10-23, 12-23, 15-23, 18-23, 18-25, or 18-30 liters) is used per 1000 grams of carriers.

In other embodiments, the volume of medium contained in the described central medium container is not less than about 6.5 liters (e.g. 3-7, 4-7, 5-7, 6-7, 6-8, or 6-10 liters) of medium per 1000 grams of carriers.

In certain embodiments, the total mass of fibrous carriers used in the described methods and compositions is at least 500 grams; or, in other embodiments, at least one of the following amounts 600, 800, 1000, 1500, 2000, 3000, 5000, 10,000, 15,000, or 20,000 grams (g), each of which represents a separate embodiment. In other embodiments, the total mass is between 500-10,000 grams, or, in other embodiments, within one of the following ranges: 500-20,000, 600-10,000, 600-20,000, 800-10,000, 800-20,000, 1000-10,000, 1000-20,000, 1500-10,000, 1500-20,000, 2000-20,000, 2000-10,000, 3000-20,000, 3000-20,000, 5000-10,000, or 5000-20,000 g, each of which represents a separate embodiment.

In certain embodiments, the total number of cells seeded in the described methods and compositions is at least 2×10⁸ cells, at least 3×10⁸ cells, at least 5×10⁸ cells, at least 6×10⁸ cells, at least 8×10⁸ cells, at least 10×10⁸ cells, at least 12×10⁸ cells, at least 15×10⁸ cells, at least 20×10⁸ cells, at least 30×10⁸ cells, between 2-20×10⁸ cells, between 2-30×10⁸ cells, between 3-20×10⁸ cells, between 3-30×10⁸ cells, between 5-20×10⁸ cells, between 5-30×10⁸ cells, between 7-20×10⁸ cells, between 7-30×10⁸ cells, between 10-20×10⁸ cells, or between 10-30×10⁸ cells.

In other embodiments, the described systems and methods enable efficient sterilization, since the individual components can be readily detached from one another and sterilized. In still other embodiments, the described systems and methods comprise single-use components, e.g. the culture vessels.

In still other embodiments, the described systems and methods enable efficient control of the cell culture conditions. In other embodiments, homeostatic control of the culture medium in the central medium container enables control of the conditions in the vessels. In still other embodiments, the flow rate is adjusted to be substantially the same for each of the vessels. In yet other embodiments, the conditions in the multiple vessels are substantially the same, by virtue of similar flow rates of medium from the central medium container.

Other embodiments of the disclosure will be apparent to those skilled in the art from consideration of the specification and practice of the present disclosure. It is intended that the specification and examples be considered as exemplary only, with a true scope and spirit of the disclosure being indicated by the following claims.

What is claimed is:

1. A cell culture apparatus, comprising: (a) a central medium container, said central medium container comprising a cell culture medium, wherein said central medium container does not contain cells; and (b) a plurality of 3-D culture vessels, wherein each of said 3-D culture vessels comprises cell carriers, comprising a 3-D matrix; wherein said central medium container is operably connected to said culture vessels via medium conduits that direct said medium from said central medium container through said 3-D matrix of said culture vessels in a parallel configuration.

2. The cell culture apparatus of claim 1, wherein said a 3-D matrix is a fibrous matrix.

3. The cell culture apparatus of claim 2, wherein said fibrous matrix is a synthetic matrix.

4. The cell culture apparatus of claim 3, wherein said culture vessels comprise living cells.

5. The method of claim 4, wherein said culture vessels are further operably connected to a means of measuring viability of said living cells.

6. The cell culture apparatus of claim 1, wherein said central medium container comprises a means of monitoring and controlling pH of said medium.

7. The cell culture apparatus of claim 1, wherein said central medium container comprises a means of monitoring and controlling dissolved oxygen concentration.

8. The cell culture apparatus of claim 1, wherein said central medium container comprises a means of collecting data on conditions within said central medium container.

9. The cell culture apparatus of claim 1, wherein said cell culture apparatus comprises a means of collecting data on transfer of fluid into and/or out of each of said culture vessels.

10. The cell culture apparatus of claim 1, wherein said cell culture apparatus comprises a means of controlling a flow rate of fluid material transferred into each of said culture vessels.

11. The cell culture apparatus of claim 1, wherein said cell culture apparatus is aseptic.

12. The cell culture apparatus of claim 4, wherein said cells are adherent stromal cells.

13. The cell culture apparatus of claim 12, wherein said adherent stromal cells are placenta-derived.

14. The cell culture apparatus of claim 12, wherein said adherent stromal cells are derived from adipose tissue or bone marrow.
